(19) United States
(12) Patent Application Publication
(10) Pub. No.: US 2016/0095877 A1
(43) Pub. Date: Apr. 7, 2016

(54) Use of Certain Foods and Dietary Supplements as Water and Beverage Activating Enerceuticals

(71) Applicant: William John Martin, South Pasadena, CA (US)

(72) Inventor: William John Martin, South Pasadena, CA (US)

(21) Appl. No.: 14/507,822

(22) Filed: Oct. 6, 2014

(51) Int. Cl. A61K 33/00; C12G 3/00; A23L 2/38; A61K 31/045; A23L 2/52

(52) U.S. Cl. CPC A61K 33/00; A61K 31/045; A23L 2/52; A23L 2/38; C12G 3/00; A23V 2002/00

(57) ABSTRACT

The Applicant has identified a biological energy pathway, which is distinct from photosynthesis and from the generation of cellular energy through normal metabolism. It is referred to as the third or the alternative cellular energy (ACE) pathway. This pathway is expressed through an energy acquired kinetic activity of water molecules. The present application extends on the breadth of compounds capable of transferring a natural force called KELEA, (kinetic energy limiting electrostatic attraction) to water and to both non-alcoholic and alcoholic beverages. They include common dietary supplements such as humic acids and common foods, such as cocoa. These products can be repeatedly used for liquid activation at very low quantities and can be removed from the liquid prior to the liquid being used for biological purposes. The studies have widespread practical applications in human and animal health as well as in agriculture. The studies also provide for a better understanding of the practice of homeopathy.

### CROSS REFERENCE TO RELATED APPLICATIONS

### Co-Pending or Previously Abandoned Patent Applications

Methods for detection of ultraviolet light reactive alternative cellular energy pigments (ACE pigments). William John Martin Submitted Dec. 24, 2007. Publication number 20090163831 Method of assessing and of activating the alternative cellular energy (ACE) pathway in the therapy of diseases. William John Martin Submitted Jan. 16, 2008. Publication number 20090181467

Enerceutical mediated activation of the alternative cellular energy (ACE) pathway in the therapy of diseases. Submitted May 8, 2008. Publication number 20090280193

Regenerative wound healing using copper-silver citrate composition. Submitted Oct. 22, 2008 Publication number: 20100099758.

Enerceutical activation of the alternative cellular energy (ACE) pathway in therapy of diseases. Submitted Feb. 11, 2009. Publication number 20090202442.

Method of using the body's alternative cellular energy pigments (ACE-pigments) in the therapy of diseases Submitted Feb. 20, 2009. Publication number 20100215763

Urine as a source of alternative cellular energy pigments (ACE-pigments) in the assessment and therapy of diseases. Submitted Mar. 5, 2009. Publication number 20100196297

Moringa oil mediated activation of the alternative cellular energy pathway in the therapy of diseases. Submitted Feb. 24, 2010. Publication number 20110208110.

Activation of the alternative cellular energy (ACE) pathway in the therapy of diseases. Submitted Jun. 9, 2010. Publication number 20110306917.

Methods for the detection of alternative cellular energy (ACE) pigments and for monitoring of the ACE pathway in the diagnosis and therapy of diseases. Submitted Jun. 13, 2010. Publication number 20110306077.

Diagnostic value of systemic ACE pathway activation in the detection by fluorescence of localized pathological lesions. Submitted Jul. 26, 2010. Publication number 20100291000

Enerceutical mediated activation of the alternative cellular energy (ACE) pathway in the therapy of diseases. Submitted July 2010.

Method of generating hydrogen in gasoline using an enerceutical product added to magnesium in a hydrogen permeable but solute impermeable container. Submitted Jul. 18, 2008. Publication number 20100011657

Energy Charged Liquids to Enhance Enerceutical Activation of the Alternative Cellular Energy (ACE) Pathway in the Therapy of Diseases. Submitted Dec. 17, 2010. Publication number 20120152755

Energy Charged Alcoholic Beverages for Enhancing the Alternative Cellular Energy Pathway in the Prevention and Therapy of Diseases. Submitted January 2011, Publication number 20120171340.

Methods for Detecting and Monitoring the Activity of Energized Water and Other Liquids Useful for Enhancing the Alternative Cellular Energy Pathway in the Prevention and Therapy of Diseases. Submitted February 2011

Methods for Increasing the Kinetic Activity of Alcohol, Water and Other Liquids, so as to Render the Liquids More Useful in Enhancing the Alternative Cellular Energy Pathway in the Prevention and Therapy of Diseases. Submitted February 2011

Methods for Increasing the Kinetic Activity of Water and Other Liquids, so as to Render the Liquids More Useful in Enhancing the Alternative Cellular Energy Pathway and in Various Other Agricultural and Industrial Applications. Submitted June 2011.

Methods for Increasing the Kinetic Activity of Water and Other Liquids, so as to Render the Liquids More Useful in Enhancing the Alternative Cellular Energy Pathway and in Various Other Agricultural and Industrial Applications. Submitted October 2011.

Use of Plants Extracts to Activate Water, Alcohol and Other Liquids. Submitted Oct. 27, 2011. Application Ser. No. 13/272,215.

Methods of Transferring Energies to Water, Alcohols and Minerals. Submitted Nov. 25, 2011. Application Ser. No. 13/304,558.

Weight Change as a Measurement of an Intrinsic Energy Property of Matter. Submitted Dec. 27, 2011. Application Ser. No. 13/340,669

Weight Change as a Measurement of an Intrinsic Energy Property of Foods and Other Materials, Submitted Jan. 5, 2012.

Heat as a Method to Enhance the Fluid Activating Ability of Humic Acids, Zeolites and Related Enerceuticals, Submitted May 28, 2014.

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not Applicable

### BACKGROUND OF THE INVENTION

As detailed in the above-listed co-pending and/or abandoned patent applications, which are herein incorporated by reference, the Applicant has identified an inducible kinetic activity of water, which relates to the water's ability to provide biological benefits beyond that of regular, non-activated water. Water activation is postulated to involve the absorption of an external force tentatively called KELEA (kinetic activity limiting electrostatic attraction). The KELEA activity of water and other fluids can be enhanced using a variety of methods, broadly categorized into two approaches. One approach involves the addition to the water of small amounts of certain unbound dipolar substances. These include humic/fulvic acids, zeolites, various other ceramics, terpenes and many others. The second approach to water activation is to place water into different types of electrical, magnetic and/or other energy fields, including that provided by previously activated fluids. In certain situations, the body itself can produce a water activating energy field, as the Applicant has now shown by including water samples near the participants engaging in Laughing Yoga classes.

Activation of water can be assessed by the differing dissolving patterns of particles of neutral red dye sprinkled onto the surface of the water. These patterns can range from stationary particles with slowly enlarging concentric rings of dissolved dye (indicative of minimal kinetic activity of the water), to rapid linear movements of the particles in activated water. The linear movements typically have a to-and-fro quality and can lead to long streaks of dissolved dye. In highly activated water, grouping of particles can collectively move as a rapidly rotating, horizontal, cylindrical vortex. While dissolved particles of neutral red dye in regular water, do not yield an ultraviolet (UV) light fluorescent solution, activated water solutions with added neutral red dye will fluoresce upon UV illumination (except if the fluorescence is quenched by particular additional components in the solution). Another useful assay to assess the degree of kinetic activation of water is to measure the rate of weight loss of capped containers of the water. For non-activated water, the weight reduction even over several hours is minimal (<0.1 mg per ml). Activated water will more rapidly lose weight, which occurs primarily by evaporation of highly kinetic molecules. These molecules show the capacity to escape in spite of the screwed cap of the container. The workable threshold of significant activity (weight loss) is >0.5 mg/ml within several hours. Values of >5 mg/ml have been achieved. The increased vaporization is essentially a measure of reduced intermolecular hydrogen bonding of the water molecules and can also be also measured as an increase in vapor pressure within completely sealed containers.

Activation can be shown with other drinkable fluids, including non-alcoholic and alcoholic beverages. High ethanol content alcoholic beverages have a higher baseline of activity than does water and non-alcoholic beverages, but can be induced to still much higher levels of activity. It has also been noted that a small quantity of activated fluid added to regular fluid will induce activation of the entire fluid in a time dependent manner. This type of progressive activation is typically achieved using 10 fold dilutions. It is similar to the procedure used in preparing, so called, homeopathic formulations. (The Applicant has learned through experimentation that it is preferable to allow for a day or so time delay between dilutions.)

Unlike, the misleading principle that homeopathic formulations have a specificity of action under the “Law of Similars,” activated water and/or alcohol can potentially provide substantial clinical benefits for a wide range of illnesses. The clinical benefits occur through the enhancement of the body's alternative cellular energy (ACE) pathway. This can be achieved through the consumption or injection of the activated fluid. It can also occur by placing the fluid in close proximity of the body. Included in this latter approach, is the addition of a small quantity (−0.1 mg/ml) of neutral red dye into the activated water or ethanol, with the solution then being illuminated with a UV light (for example a Halco 13 watt condensed UV light bulb) for 30-60 minutes. Activation of the ACE pathway is shown by the occurrence of UV inducible fluorescence within patchy areas the patient's skin and/or within the oral cavity.

There are increasing clinical data supporting the proposal that enhancing the ACE pathway can help compensate for illnesses characterized by an insufficiency of cellular energy derived from food metabolism. These conditions include inadequate oxygen from chronic obstructive pulmonary disease (COPD); impaired blood supply from cardiovascular and cardiovascular diseases; and increased energy demands due to infections and during wound healing. Of further interest is the indication that the ACE pathway plays a more unique role, beyond that of food calories, in supporting human cognition and mood.

This new paradigm has placed special importance on increasing the efficiency and availability of ways of enhancing the ACE pathway. It has also stimulated inquiry into the physics of the proposed KELEA force. The working hypothesis is that KELEA is attracted by free (unbound) electrical charges and may be fundamental in preventing the fusion and possible annihilation of opposing electrical charges. It is envisioned that certain dipolar (dielectric) compounds can capture and then release (transmit) KELEA in an oscillating manner, such that nearby electrostatically bonded water molecules will undergo a degree of charge separation. This loosening of intermolecular hydrogen bonding can be sufficient to enable the separated water molecules to function as direct receivers of KELEA. The body water includes both intracellular and extracellular water, with the latter including blood, lymph and cerebrospinal fluid.

### BRIEF SUMMARY OF THE INVENTION

The invention underscores the distinction between the use of various dietary products as sources of calories and/or biochemical nutrients and their discovered function of much smaller amounts being able to activate water and both non-alcoholic and alcoholic beverages. The patent application does so by describing water and beverage activation by a wide range of products, including certain dietary supplements, pharmaceutical compounds and natural foods. Included in the later is cocoa, an ingredient used to make chocolate. Once the water or beverage is activated, the activating product can be substantially removed by progressive dilutions (as in the practice of homeopathy) or completely removed by highly efficient filtration. The requirement for very small amounts (<1 mg/ml) of activating product is emphasized since it is often more efficient than using larger amounts (>10 mg/ml) of product. These findings will likely result in the widespread adoption of using activated water for human and animal consumption, as well as improving the efficiency of agriculture and of certain water based industrial applications. The terms “enerceuticals”; “waterceuticals”; “alternative cellular energy”; “ACE Water” and “KELEA Activated Water” convey the essence of the invention.

### BRIEF DESCRIPTION OF THE DRAWINGS

Not Applicable and none included

### DETAILED DESCRIPTION OF THE INVENTION

To formally establish that water itself is being activated, many of the water activation studies described in the Applicant's earlier patent applications using tap water have been repeated using distilled water (e.g. Arrowhead Distilled Water supplied by Nestlé). The focus of this Application is on the successful use of dietary supplements and certain foods, including cocoa, as practical means of activating water and both non-alcoholic and alcoholic beverages. The level of water activation is primarily assessed by progressively weighing the treated distilled water in capped, but not completely sealed 1 oz glass containers. (A 200 gm electronic digital Sartorius balance reading to 0.1 mg is used in most weighing experiments.) Both the rate and extent of weight loss occurring over several hours and in some experiments over days and even over many months have been made. (As noted previously, while the weight of containers with regular water remains essentially stable [<0.1 mg/ml loss over several hours]; the weight of containers with activated water will exceed >0.5 mg/ml over the same period of time and may typically continue to >1.0 mg/ml within 24 hours.) This approach can be followed by testing for the ability of ˜10% of the activated water to induce significant weight loss in a secondary container with 90% added regular distilled water.

The other major parameter of water activation is testing for dynamic linear and to-and-fro dissolving patterns of sprinkled neutral red dye particles, which remain on the surface of the water. This dynamic pattern contrasts with the slowly, concentrically spreading dissolving pattern seen when neutral red dye is sprinkled onto inactive water. (An exception to this test is when the surface tension of the activated water is so reduced that the neutral red dye particles become submerged and the dissolving dye tends to be less linear). Another useful criterion of activation is UV light fluorescence of solutions of activated water with dissolved neutral red dye (˜0.1 mg/ml). The fluorescence does not occur with solutions of inactive water and dissolved neutral red dye.

Absolute ethanol and very high alcohol content beverages, such as Stroh Rum (80% ethanol) or EVERCLEAR grain alcohol (75.5% ethanol) will naturally show a dramatic dissolving pattern of neutral red dye and fluorescence of solutions with neutral red dye. It proceeds to a much greater extent than that of even highly activated water. Yet these activities, as well as measurable weight loss in closed containers, can still be greatly enhanced using various dipolar materials. Moreover, when dissolved humic acid is added, there is a slow formation of bubbles, which rapidly collapse.

The range of compounds able to activate water and ethanol containing fluids is ever increasing. Moreover, the efficiency of humic acids, zeolites, kaolin and other ceramics in activating either water or beverages can be significantly enhanced by heating to ˜1,000° C. or higher in either a vacuum furnace or an inert gas filled furnace. (The heating is intended to disrupt covalent bonds in favor of unbound electrical charges). A method practiced over the last several months with supporting clinical evidence of efficacy has been the activation of Stroh Rum in a 500 ml bottle using 50 mg of humic acid, which had previously been subjected to an hour of heating at 1,000° C. Eighty (80) ml aliquots of activated rum (80% ethanol) are removed at various times and diluted to 800 ml with distilled water, to achieve an 8% ethanol solution. Eighty (80) ml of this diluted solution is further diluted 10 fold the next day to achieve a 0.8% ethanol solution and on the next day diluted 2 fold to reach an acceptable 0.4% ethanol concentration. This material clearly tests positive in assessments of its kinetic activity, benefits of drinking, testing on plants, etc. Several layers of Glad “Cling Wrap” (Oakland Calif.) are used to help seal the tightly screwed capped containers, when not being used to dispense activated liquid. The containers of activated water are stored separately from water samples designated as control water in various testing procedures. Otherwise, the activated solutions can lead to partial activation of the nearby water.

The amounts of products required for water activation using heated humic acids can be less than the 0.1 mg/ml used in the above illustration. Moreover, the removed 80 ml aliquots of activated rum can be replaced with fresh rum as it is being used, without an overall loss of activity over time. As with other particulate materials being used for liquid activation, the humic acids can be reused, if recovered from the activated fluid by sedimentation or by filtration (discussed later).

Various soluble pharmaceutical products have also been shown to have water-activating properties, including small amounts of procaine, Lidocaine, niacin and Dilantin. So too can many tinctures used in the formulation of homeopathics (available from Biorin Corp. Newtown square, Pa.). Of the foods tested, extracts of leaves and/or seeds of moringa oleifera trees are effective as are leaves and stems of the Ashitaba plant. HB-101, a Japanese terpene-rich tree sap extract and d-limonene, which is derived from orange peel, can also readily activate water. Certain fermenting bacteria and yeasts also produce water activating components, but their safety has not been established.

Of special interest because of its ready acceptance by the public, is the discovery of the quite remarkable water-activating activity of cocoa (cocao), as shown by using as little as 0.1 mg/ml of store purchased, packaged material. (Indeed, it is less effective and only active over a short period of time when large quantities (>10 mg/ml) of cocoa are added to distilled water. Cocoa is mainly derived from the fruit seeds of the Theobroma cacao tree, but can also be extracted from kola nuts and from certain tea plant leaves. Its major ingredient is theobromine, a dipolar molecule. The cocoa products tested included 8 oz and 8.8 oz packages of Chirardelli Premium Baking Cocoa and Droste Cocao, respectively. Given the small amount of cocoa required, each single package could potentially directly activate over 2,000 liters of water or beverage. A clue to the potential water activating properties of cocoa and of certain other herbal extracts is reports of their broadly based medicinal benefits. While others have attributed these benefits to the products' biochemical properties, the Applicant has discovered their biophysical capacity for activating liquids.

Proof that the beneficial activity of various natural products is intrinsic to their ability to activate water and not due to their biochemistry is provided by several lines of evidence: i) comparable activation of water is achievable using external energy delivering devices; ii) the level of activity tends to be inversely related to the amount of added component, with the exception of insoluble pellets; iii) the activity is retained through several 10 fold dilutions, as practiced in homeopathy; iv) activity is retained after all of the added material is removed by filtration. A simple demonstration of the latter is obtained by using the Zero Water device manufactured by Zero Technologies, 4510 Adams Circle, Bensalem Pa. 19020, to ensure removal of the water activating components; v) the levels of water and beverage activation can steadily increase over several months if the activated liquids are maintained in tightly sealed containers and vi) sealed containers of activated liquids can be used to activate nearby fluids, without any need for direct mixing of the two liquids.

Attributing the beneficial effects of using these compounds to the physics of fluid activation stands in sharp contrast to the generally accepted notions of their biological functions. For example, humic/fulvic acids and zeolites are commonly regarded as sources of bioavailable minerals. Several of the listed products are regarded as powerful anti-oxidants. Niacin is identified as a vitamin, while procaine, Lidocaine and Dilantin are thought to function as inhibitors of neurotransmission. Homeopathic tinctures are viewed to act selectively in treating the same sets of symptoms as are inducible when administered in larger quantities to normal individuals (Law of Similars). More recent concepts relating to many of the compounds include their possible intracellular influences upon gene expression (epigenetic effects). These effects can arguably lead to the increased production of certain beneficial hormones and neurotransmitters, e.g. endorphins in the case of cocoa containing chocolate.

The basic discovery is that various naturally occurring materials, including cocoa, can be used in small quantities to activate fluids. These products can seemingly act as antennas to capture an environmental energy, tentatively called KELEA. Possibly, via an oscillatory mechanism, some of these compounds can undergo some adjustment so as to release the absorbed energy, which can, thereby, be transferred to nearby fluid molecules. The transmitted energy reduces the intermolecular hydrogen bonding of the fluid molecules, potentially exposing the separated charges to the direct absorption of KELEA. This can lead to further activation of the fluid over time, as has been repeatedly seen. Once this phase of continuing further activation is achieved, the remaining presence of the initial dipolar material is no longer required and can be removed by dilution or by filtration. The recovered materials can be reused. The activated fluids can also be used to activate added fluids, including water. Fluid activation can, therefore, be a highly efficient, inexpensive, process with essentially unlimited potential.

The body can also produce KELEA absorbing materials, termed ACE pigments. They can be fluorescent, especially in the presence of certain dyes including neutral red dye. They are occasionally magnetic and can show alternating attraction and repulsion attraction when suspended in water. Chemically, they include various aromatic structures and can bind to various minerals. They form within cells, but may also form abiotically, along with the synthesis of lipid membranes and other structures.

It is also likely that electrical charges within the body can act as KELEA absorbing materials. These can include coordinated electrical activity of skeletal muscles, heart and brain. Furthermore, there may be a complementary, positive feedback relationship between activated water and electrical activity of organs in the absorption and spreading of KELEA. This is suggested by the capacity of laughter to activate water and the ability of activated water to elevate the mood of individuals.

It is reasonable to suppose that KELEA can also be diverted from its role in activating water. Microwave of homeopathic formulations is stated to diminish its beneficial activity. Compounds could absorb KELEA and either not release it, transfer it into a non-usable energy, e.g. heat. It is certainly possible that stress in some way can act as a drain on KELEA. Conceivably, it could be fast beta brain waves, tachycardia, cortisol, adrenaline, etc.

The invention now being described, it will be apparent to one of skill in the art that the findings are reflective of a new paradigm. The appended claims represent a rather narrow series of immediate practical applications of the findings. Modifications and extensions of these claims will undoubtedly follow without departing from the spirit and scope of the invention.

1. The use of products in a manner that is able to impart kinetic activity to a liquid by direct contact with the liquid, wherein once the kinetic activity is imparted to the liquid, the level of this activity will continue to increase over time, even following the complete removal from the liquid of the product(s) initially used to impart the kinetic activity.

2. The method of claim 1 in which the product used to impart the kinetic activity to the liquid is normally regarded as a dietary source of calories and/or other nutrients.

3. The method of claim 1 in which the product used to impart the kinetic activity to the liquid is used at concentrations less than 10 mg/ml of liquid, and preferably less than 1 mg/ml.

4. The method of claim 1 in which the product used to impart the kinetic activity to the liquid is humic acid.

5. The method of claim 1 in which the product used to impart the kinetic activity to the liquid is zeolite.

6. The method of claim 1 in which the product used to impart the kinetic activity to the liquid is Lidocaine.

7. The method of claim 1 in which the product used to impart the kinetic activity to the liquid is derived from the moringa oleifera tree.

8. The method of claim 1 in which the product used to impart the kinetic activity to the liquid is cocoa.

9. The method of claim 1 in which the liquid is water.

10. The method of claim 1 in which the liquid is a non-alcoholic beverage.

11. The method of claim 1 in which the liquid is an alcoholic beverage.

12. The use of an activated liquid, obtained by using the method of claim 1, to directly impart kinetic activity into a liquid, such that the level of the kinetic activity of the combined liquids will continue to increase over time.

13. The method of claim 12 in which the activated liquid, obtained using the method of claim 1 is ethanol or a high-concentration ethanol containing alcoholic beverage.

14. The use of an activated liquid, obtained by using the method of claim 1, to indirectly impart kinetic activity into a liquid by being placed in close proximity to the liquid to be activated.
